(19) United States
(12) Patent Application Publication
(10) Pub. No.: US 2024/0188906 A1
(43) Pub. Date: Jun. 13, 2024

(54) Platform, System, Device, and Method for Remote Clinical Communication

(71) Applicant: Ombumed Inc., Provo, UT (US)

(72) Inventors: Claudio A. Seoane; Marcelo Martin Seoane, Buenos Aires (AR); Mateo Miccino De Melo, Buenos Aires (AR); Walter J. Jerusalinsky, Buenos Aires (AR)

(21) Appl. No.: 18/553,547

(22) PCT Filed: Nov. 24, 2021
(86) PCT No.: PCT/US21/60864

§ 371 Date: Sep. 29, 2023

(30) Foreign Application Priority Data
Mar. 29, 2021 (AR) 1323821

(51) Int. Cl. A61B 5/00; A61B 1/00; A61B 1/06; A61B 1/227; A61B 5/0205; A61B 5/024; A61B 5/1455; A61B 5/31; A61B 7/04; G16H 40/67; G16H 80/00

(52) U.S. Cl. CPC A61B 5/7465; A61B 1/0684; A61B 1/227; A61B 5/0006; A61B 5/002; A61B 5/0022; A61B 5/0075; A61B 5/02055; A61B 5/14551; A61B 5/31; A61B 7/04; G16H 40/67; G16H 80/00; A61B 1/00103; A61B 5/02433; A61B 2560/0214; A61B 2562/0271

(57) ABSTRACT

An arrangement, a wireless interactive remote clinical examination platform device, and an instrumented platform assembly that creates an interactive remote clinic that enables direct and interactive medical remote consultations in real time with guidance from physicians and/or healthcare professionals. The invention offers an enabling technology for a comprehensive clinical examination without risk of infections in home/school/work primary care, as well as for the monitoring of remote hospitalization, interdisciplinary interconsultation and training and qualification of health professionals. The invention also comprises the method for carrying out the remote clinical medical examination by means of the arrangement and the assembly.

### BACKGROUND

The present invention relates to an arrangement, a wireless interactive remote clinical examination platform device, and a platform assembly with instrumentation that creates a remote clinic that allows direct and interactive medical consultations in real time remotely with the guide of doctors and/or health professionals. The invention offers a technology that enables a comprehensive clinical examination without risk of infections in home/school/work primary care, as well as for the monitoring of remote hospitalization, interdisciplinary interconsultation and training and qualification of health professionals. The invention also comprises a communication method and a method for carrying out a remote clinical medical examination by the aforementioned elements.

### BACKGROUND OF THE STATE OF THE ART

Before the present invention was created, technology had provided multiple independent medical devices that, being controlled by the patient himself, collect partial data in atomized form for later transmission. Technologies such as “wearables” collect partial information and accumulate it for later delivery or for automated evaluation. Other embedded and portable devices emphasize automation and the use of artificial intelligence to obtain a diagnosis or guide the use of the device. All current devices leave the use and control of the different measurement sensors in the hands of the patient and thus lack the value and effectiveness that can only be obtained through the judgment and supervision of an experienced medical professional who controls at all times the capture of medical data and use of the device.

U.S. Pat. No. 8,953,837B2 and US20190059728A1 (TYTO CARE Ltd.) disclose a portable medical device comprising one or more sensors that allows the patient to perform a self-assessment guided by predefined reference data. By means of said data, the device helps the operator of the device in the positioning and correct use of the sensors. The data obtained from the self-measurements is recorded and sent to a remote server and can be shared with a healthcare professional. The cited document is based on the concept that billions of consultations are made annually, that life expectancy increases year by year and that the amount of human resources trained to meet that demand also decreases, which is why it is necessary to optimize and facilitate the collection of clinical data to streamline the health care process and focus it through self-examination.

Document US20200286600A1 (HealthyIo Ltd.) describes a small, portable device with the capacity to measure, record and store vital signs with Bluetooth® connection to a portable processor and bases its importance on facilitating self-measurements several times a day for self-monitoring or, eventually, to save doctors or nurses time before a face-to-face consultation. The document focuses its potential interest on the loading of medical data, its storage and subsequent analysis, and focuses on self-care.

US20180192965A1 (Medwand Solutions Inc) discloses a portable home device for measuring and reporting vital physiological patient data via telemedicine. The Integrated Medical Device is a personal, portable medical monitor that provides multiple critical vital sign data for real-time face-to-face communication with qualified healthcare professionals remotely. It is also linked to a secure medical record of the patient so that the patient and/or healthcare professional can collect, archive and track information and trends.

The various devices mentioned above allow a more advanced telemedicine consultation and with greater value than a simple video call. However, all the control and management of the different measurement instruments depend on the patient who, in most cases, does not have the appropriate training of a physician and relies solely on reference images or videos for guidance.

In the timeline that shows the evolution of telemedicine, an early stage of telephone or video call consultation may be seen, to which in some cases was later added the possibility of having some sensors that accumulate information, and at a later time sending files for evaluation. The interpretation of this information with artificial intelligence systems may be foreseen in the future.

The present invention allows the collection and interpretation of clinical information interactively using the appropriate clinical instruments, and direct consultation with the medical professional who directly and instantly collects the clinical information vital for the diagnosis, optimizing the records obtained by being able to supervise and regulate the clinical instruments used, allowing to apply their experience to obtain information with non-automatic methods and maintaining the therapeutic effect that implies the preservation of the doctor-patient relationship regardless of the distance.

The object of the present invention is to allow the doctor to carry out a consultation with a clinical examination regardless of distances, in the same way as during a face-to-face consultation, giving the health professional control of the different examination instruments and continuous visualization of the results. In this way, the doctor can be sure of the quality of the measurements obtained and can receive better information for making a diagnosis by modifying the different filters, parameters and controls of each measurement during the consultation, or by asking the patient to perform certain activities during measurement such as deep breathing, coughing, or exertion. At the same time, any artificial intelligence-guided self-measurement device or simply reference data cannot detect problems that require sequential auscultation of different parts of the patient's body to follow the origin of certain pathologies in order to reach a complete and accurate diagnosis. Clearly, the lack of control and supervision during auscultation can lead to a wrong diagnosis.

The major difference that the present invention offers compared to other prior art arrangements is that those are mostly devices or software for videoconferencing or self-examination devices that record in isolation, by means of some sensors, some parameters and then send delayed information. On the other hand, the present invention enables the physician to carry out a comprehensive and independent clinical examination, regardless of the geographical distance, and dynamically interacting with the patient and controlling the clinical instruments for their optimal use based on their professional knowledge, thus replacing a face-to-face consultation. The participation in the chain of diagnosis of specially trained personnel is an alternative option provided by the present invention, which is based on the resolution of medical problems, regardless of the location or type of urgency, without intermediaries between doctor and patient.

The present invention enables immediate real-time interaction of the medical examination, allowing the examination and collection of clinical information provided by the instruments incorporated in the clinical examination platform device to be adapted to the current pathology of the patient, or their clinical condition, allowing obtaining data records in unconventional ways; for example, auscultating peripheral vessels along the entire course of an artery to search for a local murmur or a vein to detect the presence of an arterio-venous fistula or different places in the chest to diagnose the spread of a murmur according to the origin and anatomical alteration that originates it. In one situation, it may be possible to investigate which of the mitral valve leaflets is malfunctioning, analyzing the direction of propagation of the murmur, or verifying inter-chamber communications, persistent ductus, etc. Likewise, the action, for example, of a pediatrician when examining a child with an acute bronchial condition with the present invention would enable the professional to obtain auscultation of signs of airway spasm and/or poor O₂ saturation and evaluate the response when taking a bronchodilator medication and, based on this, he may determine the need or not to go to a potential hospitalization center. This capacity for interactive consultation dynamics allows the development of a clinical analysis as the one carried out in a face-to-face consultation, which is not possible using other similar delayed action inventions, nor through consultations through simple video conference or chat.

The present invention is intended for personal or family use or for a work group, with tools to identify each individual within the group and connect them through the creation of a remote clinical office to carry out a clinical evaluation, in which evaluation instruments are available for this purpose. A cell phone with an APP software are also used to establish visual and auditory communication and interaction among the doctor and the device of the invention by means of a WiFi connection to the cloud, and after having identified it, establishes a secure direct “peer to peer” communication to make the query. The “peer to peer” embodiment is managed by the interactive remote clinical examination platform device (IRCEPD) on the patient's side in order to guarantee speed, privacy, security and data economy for cases of slow access connectivity. If blocking by firewalls happens, the platform can also establish an alternative connection relay in order to ensure all technical conditions. The clinical examination platform device located at the patient's site has the electronic resources, instruments and clinical software necessary for the clinical examination, an internal CPU, an operating system with permanent self-update capacity and reconnection mechanisms in case of communication failure. The clinical examination device also has a backup battery and a wireless recharging system for it, so it does not require physical connection to any other device at any time. This provides electrical safety, immunity against interference, and increased wearing comfort.

The examination platform on the doctor's side can be shared in order to generate a space for consultation with another specialist doctor or also with several doctors to carry out conferences, discussions or clinical training sessions for “fellow” doctors (in medical jargon it refers to doctors in rotation or doing an internship or specialization), medical students or others.

### SUMMARY

The present invention refers to an arrangement that allows creating a remote clinical examination office where a shared consultation space is established between a doctor and a patient by means of an assembly comprising a remote clinical examination platform device, for the performance of a immediate or interactive medical consultation, providing a remote consultation space with all the necessary instruments for the examination and clinical auscultation. The invention offers an enabling technology for conducting a comprehensive real-time clinical examination without risk of infection in primary home/school/work or travel care and remote hospitalization monitoring.
a) A platform assembly at the patient's site comprising an interactive remote clinical examination platform device (IRCEPD) that holds multiple clinical assessment instruments and contains a firmware for the electronics and a software layer for immediate communication, interactive operation of the device and, simultaneously, channels of clinical data.
b) An equipment having a “frontend” software for letting the doctor enter into the remote office session in real time, for handling the device, for consulting and storing new data and records into the patient's medical file.
c) A cell phone equipped with a management and communication mobile phone application software (APP) for the patient.
d) An equipment having a “backend” software for data processing and storage of digital medical records.

In software design, the “front end” is the part that interacts with users and the “back end” is the part that processes the input from the “front end”. The separation of the system into “front ends” and “back ends” is an abstraction that helps to keep the different parts of the system separate. The general idea is that the “front end” is responsible for collecting the user's input data, which can be of many and varied forms, and transforms them and adjusting them to the specifications demanded by the “back end” in order to process them, returning generally a response that the “front end” receives and shows to the user in an understandable way. The connection of the “front end” with the “back end” is a type of interface.

In this document, starting from this paragraph and when referring to the “front end”, the text will be referring to the computer equipment that stores and runs the “front end” software and, when referring to the “back end”, the applicant will be referring to the computer equipment that stores and runs the “back end” software.

The present invention comprises an assembly that includes an interactive clinical examination device on the patient side comprising hardware with clinical instruments, a clinical software that performs calibration, acquisition, processing and interactive communication and control, an APP type software for cell phones also on the patient's side, which also allows the visualization and face-to-face dialogue between doctor and patient, a communication management backend that performs the authentication of the parties involved (patient, doctor and device) that establishes peer to peer communication, and a web application on a terminal (Ipad, laptop, PC, Tablet or cell phone) on the doctor's side that allows the creation of an interactive remote office from where the space for consultation and interactive remote clinical medical examination is established, regardless of the distances, recreating the environment of a physical office. As was said before, the doctor's side can be shared in order to generate a space for consultation with another specialist doctor or also with several doctors to carry out conferences, discussions or clinical sessions for training fellow doctors (that is, doctors in rotation or doing an internship or specialization), medical students or others.

In a basic embodiment, the arrangement of the invention has 3 basic parts: (i) the assembly comprising the device at the patient's site with all its clinical instruments, (ii) a cell phone equipped with a cell phone application (APP) and (iii) a equipment running a web application software for the doctor. When the patient activates his cell phone application, it immediately generates a QR code image that, when faced with the camera of the interactive clinical device, it identifies it, and the communication generated by an office is established for the doctor to access the web application (See FIGS. 16 and 17).

The device at the patient site is portable, it has rechargeable batteries with a wireless charging base and can be carried anywhere having access to connectivity where the patient requires it.

The problem solved by the present invention is to avoid that patients have to travel to the place of medical care, with the costs, diversion of activities, schedule problems, and the risk of infection in a hospital environment that these imply. In cases of remote areas, patients end up desisting from being treated in the traditional way. The present invention modifies the form of clinical care, creating a new space for consultation and treatment, solving the problem of the state of the art generating the space of a clinical care office regardless of distance, by using the device of the present invention with clinical instruments, software, and a secure communication mechanism that generates a remote office that can be in the same physical environment or in a distant one, regardless of distances.

### EXAMPLES

Medical emergencies with difficulty in accessing quick professional care due to: distance to health centers, time frame, insecurity, lack of transport means, not having company or help.
Fear of going to health institutions due to the possibility of contagion.
Turning up of symptoms in places or moments of difficult access to health professionals such as schools, offices, prisons, long-distance transport or remote places.
Travel illnesses with the need to consult a trusted professional and without language barriers.
Need for guidelines for the monitoring of chronic diseases and encouragement to carry out self-controls.
Anguish generated by the turning up of feelings of physical discomfort at times when it is difficult to interrupt a work or social activity.
Difficulty obtaining a second opinion when faced with a worrying diagnosis.
Time wasted in going to a doctor's office to obtain a prescription, order of studies, deliver a result or evaluate the evolution of a pre-existing pathology. Given the need for a face-to-face consultation, difficulty in establishing the urgency and the choice of the institution and specialty.
Comfort, convenience and ease to carry out a clinical consultation.

Other aspects and advantages of embodiments of the present invention will become apparent from the following detailed description, taken in conjunction with the accompanying drawings, illustrated by way of example of the principles of the invention.

The present invention has an interconnected set of validated clinical instruments (which require regulatory controls in accredited laboratories and regulatory agencies), and technological services in a shared doctor-patient space independent of distance through which people can be remotely and securely treated by doctors, in order to achieve as much as possible the benefits of a traditional face-to-face consultation, namely:
Establish a face-to-face communication between the patient and the doctor that helps consultation and questioning and interpersonal connection, allowing the reading of voice inflections and facial expressions as if the meeting took place in person.
Establish a mechanism so that the doctor can carry out the remote consultation, examine, auscultate and use the medical instruments (digital electronic stethoscope, thermometer, electrocardiograph, otoscope, dermatoscope, etc.) under his full control.
That the set be integrated in a flexible and fluid way with the administrative systems used by health institutions to operate shift reservations, waiting rooms, etc.
That the entire process is highly scalable and there are no “bottlenecks” with the exponential growth in the number of users.

### BRIEF DESCRIPTION OF THE DRAWINGS

FIG. 1 shows the general layout of a first embodiment of the arrangement.

FIG. 2 shows the general layout of the arrangement where a second specialist doctor participates to carry out an interconsultation.

FIG. 3 shows the general layout of the arrangement in which a group of doctors share the remote office examining the patient for case discussion in the form of a round of doctors or for training.

FIG. 4 shows the arrangement for holding an athenaeum for discussion of cases by the doctors in the office and other professionals who can be in the same place or remotely connected in turn, in a group of one or more in each remote site.

FIG. 5 shows the components of the interactive remote clinical examination platform assembly (patient's side).

FIG. 6 shows a patient using the Electrocardiogram (ECG) Function.

FIG. 7 shows the pharyngoscope of the remote clinical throat examination device.

FIG. 8 shows the interactive remote clinical examination platform device (IRCEPD) with the otoscope function.

FIG. 9 shows the remote clinical device for the dermatoscope function.

FIG. 10 shows the digital electronic stethoscope for auscultation of pulmonary, cardiac or abdominal sounds of the remote clinical device

FIG. 11 shows the contact-free infrared clinical thermometer from the remote clinical device

FIG. 12A shows the block circuit of the interactive remote clinical examination platform device IRCEPD.

FIG. 12B shows in more detail the block circuit elements that make up the interactive remote clinical examination platform device IRCEPD.

FIG. 13 outlines the communication authentication protocol.

FIG. 14 shows the signaling process.

FIG. 15 shows the “peer to peer” communication.

FIG. 16 shows a flow chart of the establishment of the Remote Clinic on the Patient's side in the method of the present invention.

FIG. 17 shows a flow chart of the establishment of the Remote Clinic on the doctor's side in the method of the present invention.

FIG. 18 shows the functional distribution of the clinical instruments of the interactive clinical examination device.

FIG. 19 shows the block circuit of the electrocardiogram (ECG) function instrument.

FIG. 20 shows the block circuit of the plethysmograph and the oximeter.

FIG. 21 shows the block circuit of the otoscope.

FIG. 22 shows the block circuit of the pharyngoscope.

FIG. 23 shows the block circuit of the stethoscope.

FIG. 23A shows the block circuit of an alternate stethoscope configuration.

FIG. 24 shows the block circuit of the spectrometer.

FIG. 25 shows the block circuit of the non-contact infrared thermometer.

FIG. 26 shows a patient using the plethysmogram and oximetry function.

FIG. 27 shows a screen of the doctor's APP during a remote interactive office session performing an electrocardiogram on the patient in real time.

FIG. 28 shows a doctor's APP screen during remote interactive office session examining the patient's throat in real time.

FIG. 29 shows a physician APP screen during remote interactive office session examining the patient's ear in real time.

### DETAILED DESCRIPTION

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the indicated embodiment is included in at least one embodiment of the present invention. Thus, the phrases “in one embodiment,” “in an embodiment,” and similar language throughout this specification may, but do not necessarily, all refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the indicated embodiment is included in at least one embodiment of the present invention. Thus, the phrases “in one embodiment,” “in an embodiment,” and similar language throughout this specification may, but do not necessarily, all refer to the same embodiment.

### Remote Office for Interactive Clinical Examination:

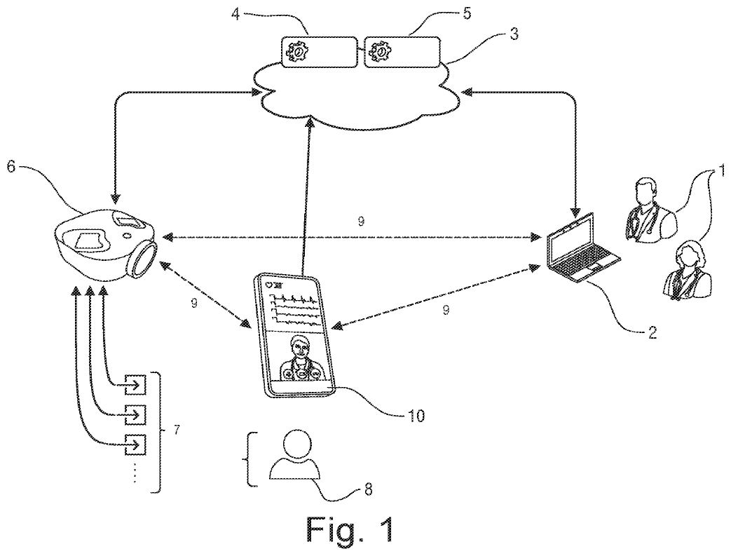

FIG. 1 shows the wireless interactive remote clinical examination platform IRCEPD 6, the cell phone 10 where the patient's APP works, the Internet network and the backend 4 in the cloud 3 that manages the authentication of patient 8 and doctor 1 and generates the keys for communication between the parties, and a doctor 1 controlling and viewing at the same time the instruments of the clinical device 6 during the performance of the medical act of clinical examination of the present invention, establishing a remote office with two acting parts: a patient 8 and a doctor 1. A general diagram of the arrangement of the present invention is shown representing the remote interactive office comprising of:

A “frontend” 2 where the doctor (s) 1 through an Ipad, a Tablet, a laptop or even a Smartphone, or through the APP, communicate over the Internet with the “cloud” 3 to request P2P signaling (peer to peer) 9, verify labor shifts, obtain authentication and have access to the patient's medical history 5. Through the Internet, the “cloud” communicates with the remote clinical examination IRCEPD 6 which, together with the clinical instruments 7 integrated in it, form the “backend” at the patient's side 8. The software installed on the “backend” initially identifies the clinical examination IRCEPD 6, validates it through private and secure procedures and thus establishes an office environment where the physician 1 instantly and dynamically views the examination result. Simultaneously, patient 8 and doctor 1 maintain visual and audio connection through a cell phone 10 on the patient's side with the “frontend” of doctor 1; all this regardless of the distances. Once the communication starts, the system registers the credentials of the doctor 1 and the patient 8 so that the consultation continues in a communication with encrypted peer to peer mode 9 for greater communication speed and security

Remote Clinical Office for Clinical Examination with Interactive Consultation:

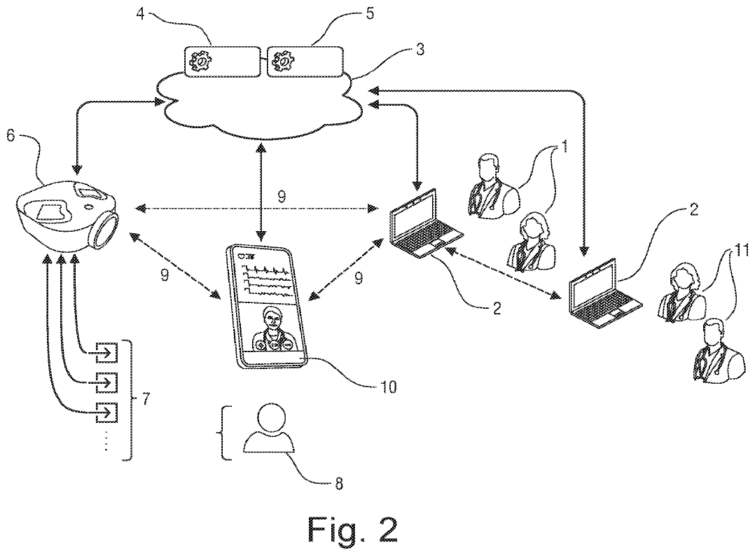

FIG. 2 shows a general diagram of the arrangement of the present invention, representing the remote interactive office for interconsultation with other professionals 11, sharing patient information interactively, allowing discussion and analysis of the case simultaneously.

### Remote Clinical Office for Clinical Examination, Training/Specialization:

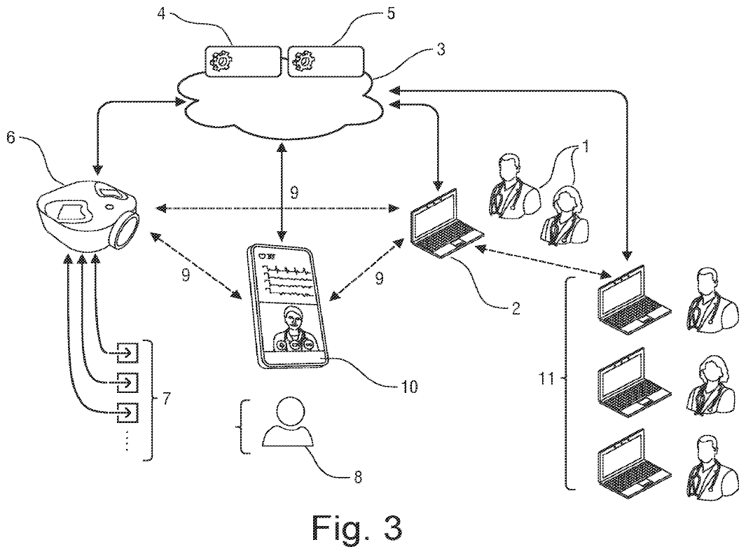

FIG. 3 shows a general diagram of the arrangement of the present invention representing the remote interactive office where an examination is carried out, where training professors, include doctors or students 11 for study and practical training, resembling the rounds of doctors in the hospitals.

Remote Clinical Office for Study and Discussion of Cases without the Patient in the Athenaeum Mode:

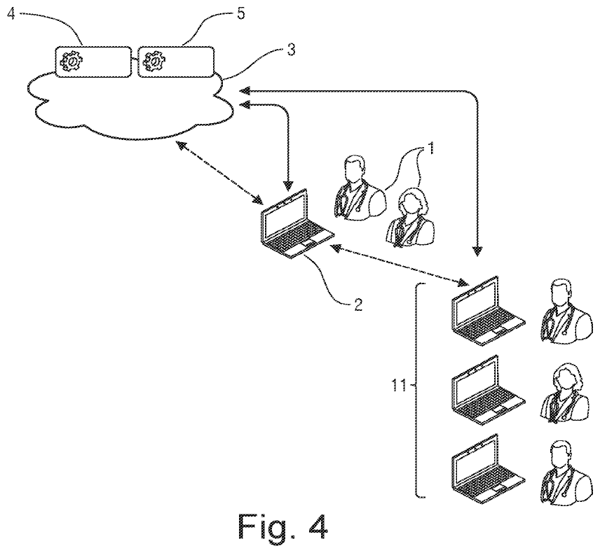

FIG. 4 shows the way a medical Athenaeum equivalent to the one carried out in person by the staffs of doctors from health institutions is held.

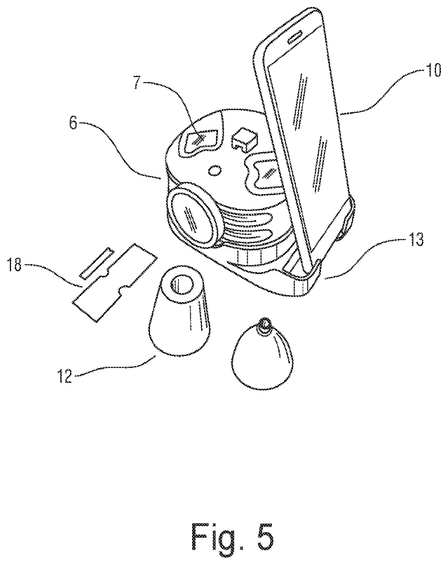

FIG. 5 shows the components of the interactive remote clinical examination platform assembly (patient's side) that comprises the IRCEPD 6, the wireless charging base 13, the cell phone 10 in the support base so that the patient can interact with the doctor with image and sound with the doctor without occupying his hands, the accessories 12 of the instruments 7 of the IRCEPD 6, the dermatoscope adapter, the otoscope adapter, and test strips 18 for rapid analysis in the real-time spectroscope.

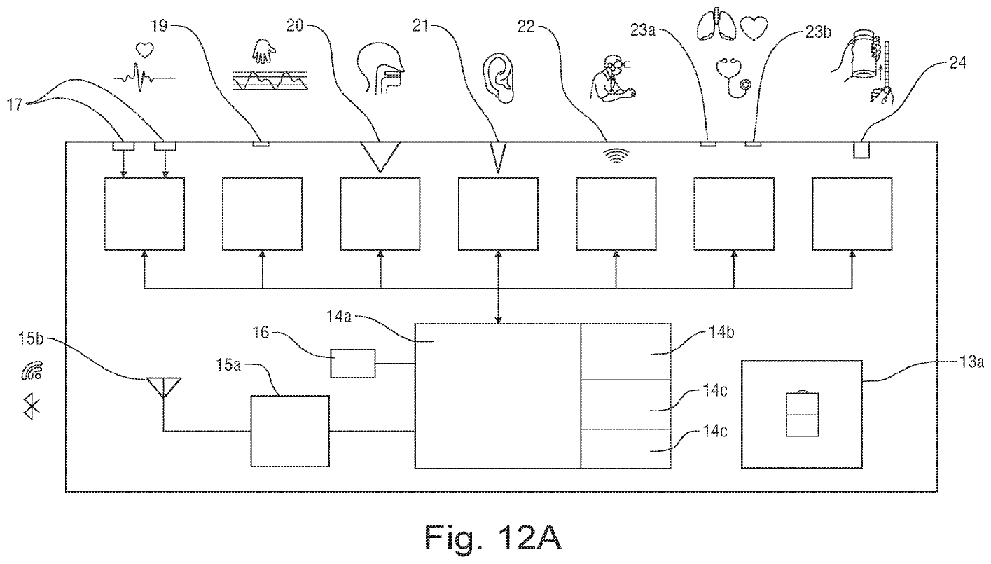

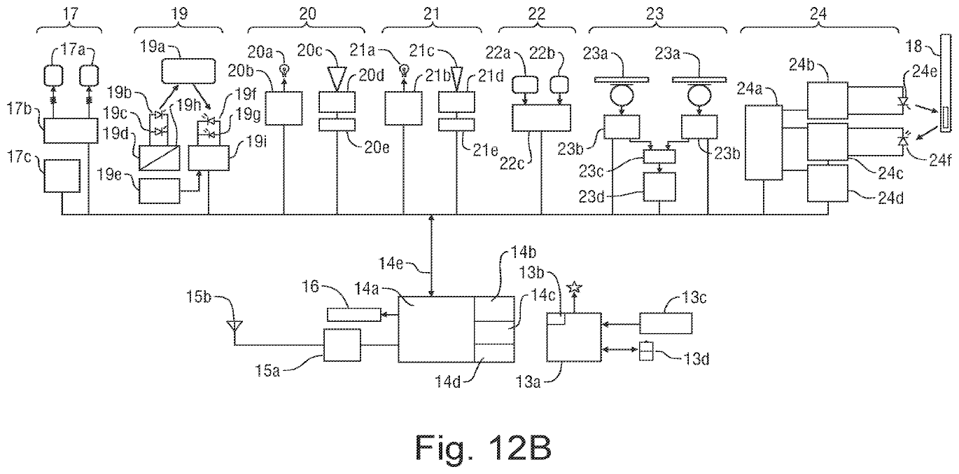

### Description of the Internal Parts of the Interactive Clinical Examination Platform Device IRCEPD (See FIGS. 12A and 12B):

The IRCEPD comprises:
Power Management Unit 13a, Wireless Battery Charger 13c, Voltage Regulators 13b.
CPU 14a with its memory 14d, operating system 14b and management software 14c for clinical instruments 7, external communication circuit via WiFi network 15a, WiFi and BlueTooth® antenna 15b.
Expansion of internal instruments 16.
Electrocardiograph (ECG) with a two electrodes connection. Includes preamps, filters, and A/D conversion 17.
Plethysmograph and pulse oximeter with LED emitters, photodetectors, optical elements and low noise electronics with ambient light rejection to obtain the plethysmography curve and oxygen saturation level 19.
Pharyngoscope and dermatoscope: Camera and optical lens for throat examination with adjustable illumination of compensated spectrum 20.
Otoscope: Camera and optics for ear examination with removable disposable/sanitizable speculum and adjustable illumination of compensated spectrum 21.
Clinical thermometer with an approach infrared body temperature sensor (non-contact) 22.
Digital electronic stethoscope with gain and ambient noise cancellation processing and adjustable filters for auscultation with specialized filters, eg: heart, abdomen, lung, vascular paths 23.
Test strip analyzer spectrometer for rapid analysis by real-time spectroscopy, having several LED light emitters and photosensors of different wavelengths calibrated to form a spectral analysis 24.

The present invention enables carrying out a medical consultation with a direct interactive comprehensive physical examination between doctor and patient, thus establishing with the remote office an integral solution with which the contact of the patient and their doctor is completed, including the necessary instruments for such a task under the direct control of the acting physician. It is of utmost importance that the health professional can control and guide the use of these devices to obtain the best clinical examination and act according to the patient's symptoms and their knowledge.

The known devices of the state of the art and some known “wearables” (devices that are “dressed” like watches or similar) allow the recording and storage of vital signs automatically, or through self-diagnostic maneuvers, which provide limited information for diagnosis, or the evolution of some chronic pathology or results of a programmed physical activity, but there are clinical facts that distinguish the operative mode carried out by the present invention.

On the other hand, the present invention generates a consultation space with clinical instruments to be operated by a professional who is thus in a position to conduct a clinical physical examination regardless of distance, with the advantage of immediacy and interaction that allows resembling a remote clinical office, also avoiding the risks of contagion, hospital infections and other health problems.

The strategic arrangement of the instruments in the device of the Interactive Remote Clinical Examination Platform (IRCEPD) of the present invention allows the practical implementation of the multiple functions in a small size device, easily manipulated allowing access to the different parts of the target body clinical examination without limitations.

As already mentioned above, the present invention comprises a medical interactive clinical examination platform device with portable instrumentation for face-to-face or remote physical clinical medical examination and consultation. This device includes clinical instruments for the evaluation of vital/clinical parameters of a patient such as: electrocardiograph, photoplethysmograph, oximeter, non-contact infrared thermometer, digital electronic stethoscope, pharyngoscope, dermatoscope, otoscope, spectrograph and others that can be added, based on the concept of the platform for remote interactive attention. In addition, the platform's clinical device is capable of receiving new clinical instruments such as blood pressure measurement, spirometry, blood glucose, reagent analysis, electroscopy, etc.

The system of the present invention comprises in its platform device the basic clinical instruments suitable for the clinical examination of a patient and these are not removable. The patient who consults can be physically present at home, in a hotel, when traveling, on a plane, or anywhere where he has Internet connectivity. The doctors can be in a hospital, a care center or anywhere else where they have connectivity.

Different configurations of the arrangement allow its use individually or in groups, families, working offices, work groups, schools, etc. The device allows the examination, auscultation and monitoring of patients by their doctor, thus resembling the conditions of a face-to-face consultation.

### Operation of the Arrangement

Peer/participant: refers to each of the devices that contain the software used by the doctor or the patient, or to the clinical interactive device.

End-to-end Encryption (E2EE): It is an encryption system by which messages can only be read by their recipients, being not decodable in all intermediate communication stages. End-to-end encryption ensures that a message encrypted by an original sender can only be decoded by its end recipient.

Server in the cloud/Backend: It is a service that is provided from the infrastructure of a ‘cloud computing’ provider, that is to say, outsourced computing resources “in the cloud”, which are featured by their great reliability, security, availability and scalability, which are accessed by the patient through their mobile APP, such as the doctor and the clinical interactive device.

Cloud: It is a metaphor that refers to the information and communication infrastructure available in high-performance computer centers distributed on the Internet.

### Single Remote Clinical Office Session:

### Stages of Communication:

The communication between the participants of the arrangement of the present invention is divided into three stages:
1. Authentication.
2. Intercommunication between participants (“Signaling”)
3. Direct communication (“Peer to Peer”)

All stages of communication use end-to-end encryption.

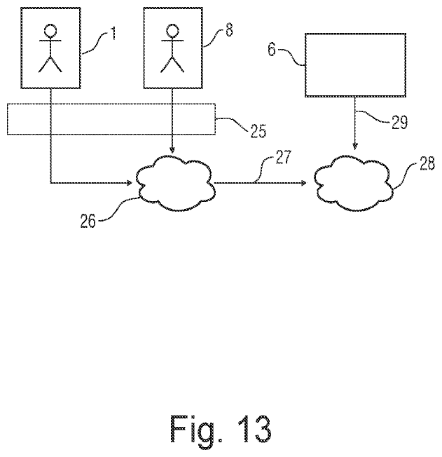

### 1. Authentication (FIG. 13):

The authentication stage is carried out by verifying the authenticity of the patient's credentials, verifying the authenticity of the doctor's credentials and verifying the identification of the IRCEPD to be used.

In the authentication step, it is validated that patient 8 is correctly registered; In this way, when the patient starts a consultation, he requests the “Cloud Server”/“Backend” to create a single remote clinical office session, where the patient passes to the next stage of “Intercommunication between participants” (Signaling).

Once the unique remote clinical office session has been created, the clinical device must scan the QR code of the session generated by the patient's App, in which it will get the necessary information to join the unique remote clinical office session, and passes to the next stage of “Intercommunication between participants” (Signaling).

In turn, in the same way, a doctor 1 who is available for the consultation, may join the single remote clinical office session, where he/she passes to the next stage of “Intercommunication between participants” (Signaling).

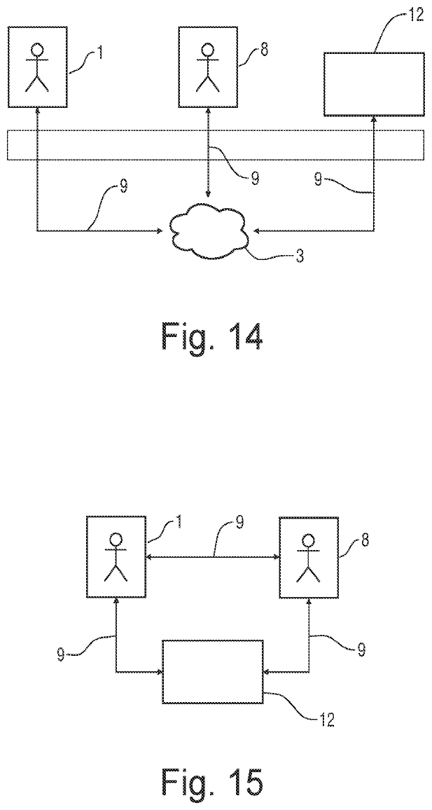

### 2. Intercommunication Between Participants (Signaling) (FIG. 14):

Signaling/Intercommunication between participants: It is the process in which each party (it can be a doctor, a patient or a clinical interactive device), exchanges the corresponding information through the server in the cloud/backend in order to carry out a “direct communication in peer to peer mode”.

From the remote clinical office session created in the previous step (Authentication), the stage of intercommunication between peers begins, in which the necessary information is exchanged with each participant through the Cloud server/backend, so that they can establish direct communication (Peer to Peer) with their respective peer, that is, the patient 8 through their cellular APP, the interactive remote clinical examination IRCEPD 6 and the doctor 1 through their APP.

### 3. Direct Communication (“Peer to Peer”) (FIG. 15):

Direct Communication/Peer to Peer: It is a communication in which each participant connects directly with another participant, without having an intermediary. Since this, in some cases, is made difficult by the action of a firewall or some other technical limitation, the initial communication is done through the Cloud Server/Backend to guarantee communication in all situations.

Other definition of Peer to Peer: A peer-to-peer network is a computer network in which all participants or devices work without fixed clients or servers, forming a series of nodes that behave as equal to each other.

Once the direct communication (Peer to Peer) has been established, the following actions can be carried out:

### Scheduled Medical Consultation:

Around the time of the consultation, the patient starts a session in the cloud server/backend using the APP application on his cell phone 10.

This operation also establishes an encrypted and secure connection 5 with the server in the cloud/backend for signaling and managing the connection in real time. The management platform of the remote clinical office begins the identification of the participants of the clinical examination to be performed (patient and doctor), proceeding to their authentication by passing the necessary credentials and then using encrypted peer to peer communication. Once the examination participants (the interactive clinical examination platform IRCEPD with its instruments, the patient, the examining physician) have been authenticated, peer-to-peer communication is performed without the intervention of other participants/processors.

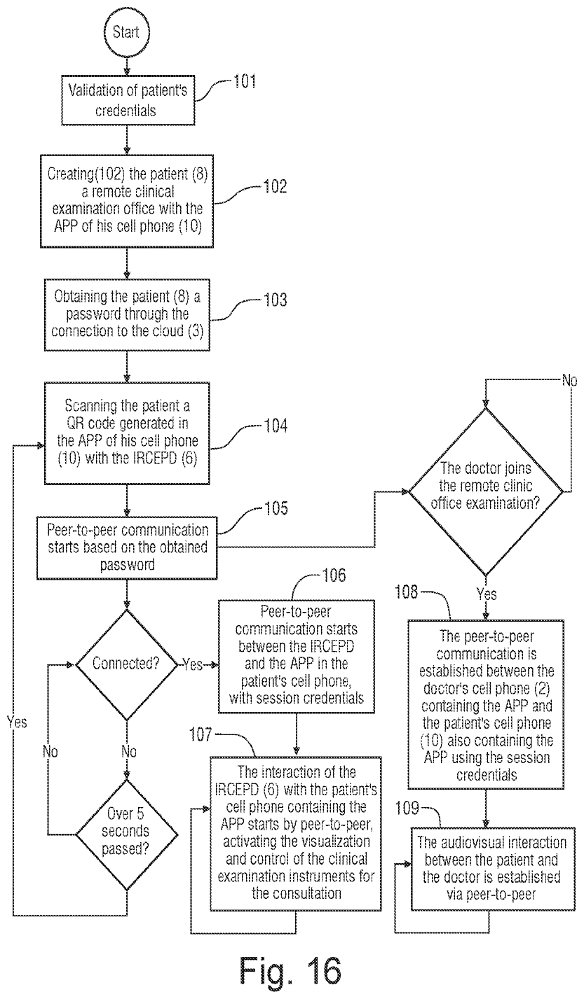

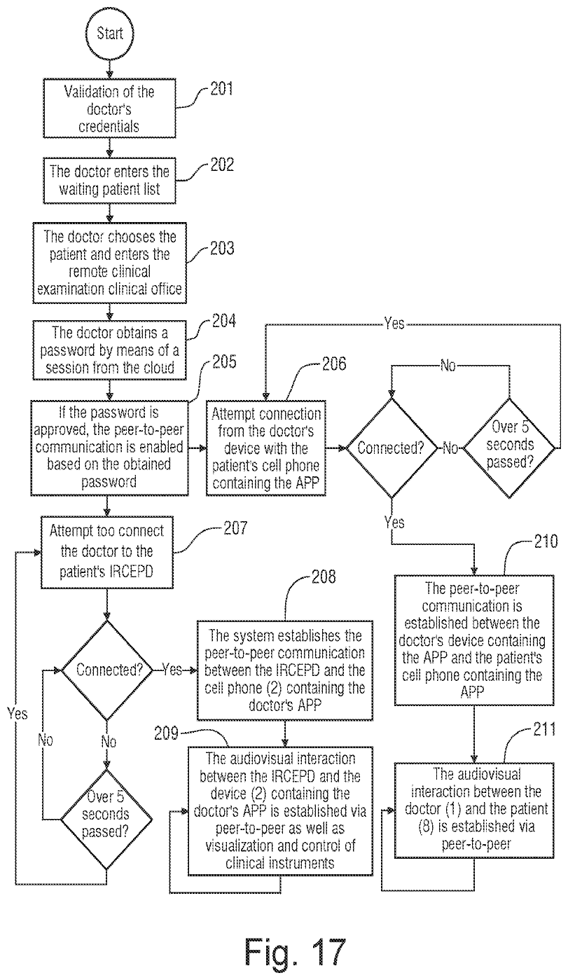

Before or immediately after, the patient turns on the IRCEPD 6, and scans the image of the QR code with information from the authenticated remote clinical office session displayed by the APP of his cell phone 10. When starting, he also initiates an encrypted and secure connection with the software of backend signaling 4. The same happens in the web application used by the doctor. See FIGS. 16 and 17.

By means of this triple connection based on the signaling service, new encrypted and secure connections are then negotiated between the three participants, which are no longer channeled through the central service, but, on the contrary, are direct between the parties, via P2P (peer to peer) 9. See FIG. 15. It is through these direct, secure and end-to-end encrypted connections 9 that the data channels of the instruments of the remote clinical examination device, electrocardiogram, are transmitted in real time: oximetry, plethysmography, temperature, pharyngoscope, dermatoscope, otoscope, spectrography, and others, as well as voice and image for the interaction of doctor 1 and patient 8, which the doctor can conveniently control and view from his screen. In addition to guaranteeing the privacy and security of the queries, this architecture ensures the scalability of the service, since the heaviest burden of voice, image and data transmission does not depend on the central servers.

In this way, benefits such as privacy, speed, scalability, robustness, decentralization, anonymity, and consequently great security are provided. In the event of any limitation or obstacle in this connection mode, such as firewalls or others intended to block communication, the management platform continues to mediate to allow the connection in all cases that may arise in different network topologies.

### Unscheduled Spontaneous Consultation:

Once authenticated in the same way as in the scheduled medical consultation, the patient sends from his mobile application APP a link to start the consultation that he sends to his doctor so that he can directly access the consultation.

### Methods of Using the Arrangement

Based on what has been explained so far, the methods of use of the provision can be summarized as follows.

### Establishment of the Remote Clinic on the Patient Side

FIG. 16 is a flow chart illustrating an example of a sequence of operations carried out for the remote clinic office setting on the patient's side. At startup, validation of the credentials of patient 8 is performed (step 101). Next, the creation of the remote clinical examination office is performed from the APP of the patient's cell phone 10 (step 102). Then, the obtaining of passwords is established by means of a session from the cloud 3 of the system (step 103). Next, the patient 8 scans the QR code generated in the APP of his cell phone 10 using his IRCEPD 6 (step 104). If the passwords are approved, the peer to peer communication 9 starts (step 105). From that moment on, the system verifies if the connection was established and, if that does not happen, it retries for 5 seconds. If the connection is not established after 5 seconds, the patient must re-scan the QR code (step 104) to retry establishing peer to peer communication 9. In turn, the system verifies if physician 1 joins the remote clinical examination office and does not move forward until that happens. On the patient's side, once the connection is established, the peer-to-peer communication is established between the IRCEPD 6 and the cell phone 10 containing the patient's APP, with the session credentials (step 106). Then, on the patient's side, the communication of the IRCEPD 6 and its cell phone 10 containing the APP by peer to peer 9 starts, and the visualization and control of the clinical examination instruments for the consultation is established (step 107). On the side of doctor 1, once he is checked, he joins the remote clinical examination office, and the peer to peer communication is established between the doctor's cell phone containing the APP and the cell phone of the patient 10 also containing the APP using the session credentials (step 108). Finally, the audiovisual interaction between patient 8 and doctor 1 is established (step 109).

### Establishment of the Remote Clinic on the Doctor's Side

FIG. 17 is a flow chart illustrating an example of a sequence of operations carried out for the establishment of the remote clinic office on the physician's side. At startup, validation of the credentials of physician 1 is performed (step 201). Next, he enters the waiting patient list (step 202). He then makes the patient choice and enters the remote clinical examination office (step 203). Next, the obtaining of passwords is established by means of a session from the cloud 3 of the system (step 204). If the passwords are approved, peer to peer communication 9 is initiated (step 205). From that moment on, the system tries to connect the IRCEPD 6 (step 207). If that does not happen, it retries for 5 seconds and if the connection is established, the peer-to-peer communication is established between the IRCEPD 6 and the cell phone 10 containing the patient's APP (step 208) and the audiovisual interaction between the IRCEPD 6 and device 2 containing the APP of physician 1 (step 209). In turn, with the start of the peer to peer communication 9 (step 205), an attempt is made to establish the connection from the device 2 of the doctor 1 with the cell phone 10 of the patient 8 that contains the APP (step 206). The connection attempt from the device 2 of the doctor 1 with the cell phone 10 of the patient 8 that contains the APP (step 206) is repeated for 5 seconds. If the 5 seconds are exceeded without success, step 206 is repeated. If the connection is established, the peer to peer communication is established between the doctor's device 2 containing the APP and the cell phone 10 containing the patient's APP (step 210). Next, the audiovisual is established between doctor 1 and patient 8.

### Basic Components of the Interactive Remote Clinical Examination Platform Assembly.

FIG. 5 shows an image of the elements that are part of the platform assembly.
Interactive Remote Clinical Examination Device (IRCEPD) 6.
Accessories 12.
Wireless battery charging base 13.
Cellular in base 10: allows the patient to manipulate the clinical examination device 6 while maintaining visual contact with the doctor.

FIG. 12A shows a diagram with a detailed description of the hardware of the interactive remote clinical examination device (IRCEPD) 6 which is comprised of a printed circuit with electronic components, a CPU and a power management system, thus forming an autonomous system.

FIG. 12B shows the block circuit of the device with the detail of the parts of each clinical instrument included in it.

### Instrumentation:

### Layout of the IRCEPD Clinical Instruments:

The present invention was carefully designed for providing the internal physical distribution of the instruments of the IRCEPD 6, obtaining an ergonomic optimization and clinical functionality of the device to achieve a small size, suitable for use with one hand and with good grip to avoid falls and breaks, as well as the comfort and practicality of use of all the clinical instruments included therein.

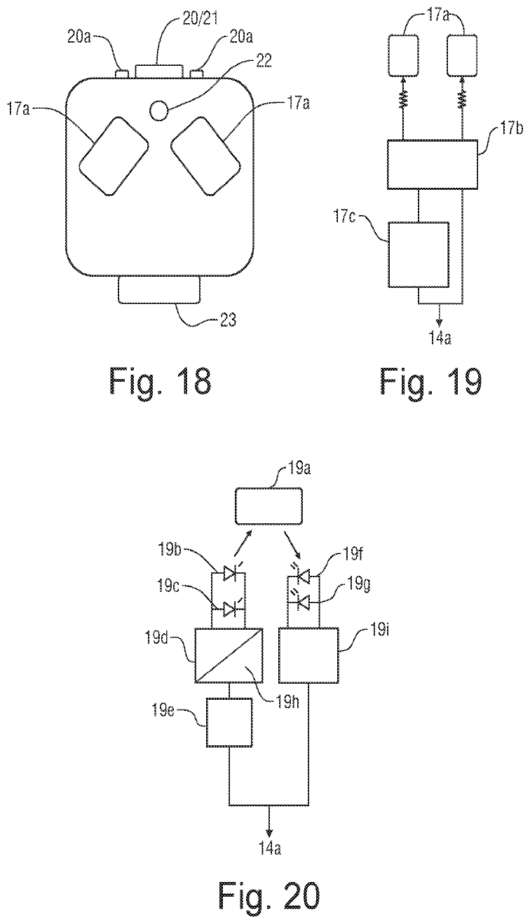

FIG. 18 shows the IRCEPD 6 comprising the following instruments: electrocardiograph electrodes 17a, illumination for pharyngoscope/dermatoscope/otoscope 20a pharyngoscope/dermatoscope/otoscope 20/21, non-contact infrared thermometer 22 and digital electronic stethoscope 23. FIG. 12A shows the general circuit diagram of the IRCEPD 6.

### Operation of the Clinical Instruments:

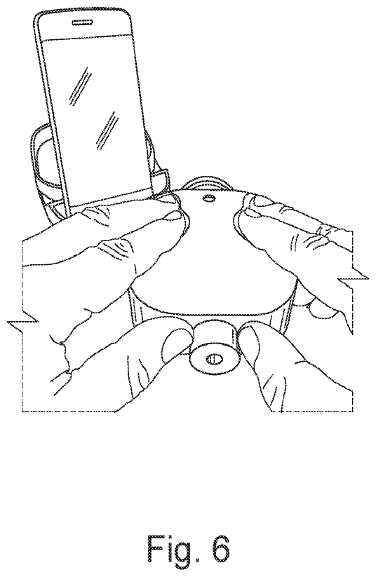

### Electrocardiograph (ECG) (FIGS. 6 and 19).

The patient sitting in a comfortable position, relaxed, resting his arms on a table, for example, places his index and middle fingers of each hand on the electrodes on the upper part of the IRCEPD 6 as indicated in FIG. 6. On the other end, the doctor immediately views the generated ECG graph on his web APP and he can change the sweep speed, gain and filter, as well as require some action from the patient (exercise, hand grip test, apnea, etc.) to achieve the desired result.

The doctor can also save these data in the patient's medical record if he so wishes. The arrangement of the electrodes allows great ease of use with arms relaxed on a table with the option to do it resting on one leg on the 3rd reference electrode when the doctor requires it depending on the clinical situation of the patient.

FIG. 19 shows that the electrodes 17a are connected to a high impedance preamplifier and programmable gain 17b that amplifies and conditions the electrical signals of the heart. The 16 bit A/D converter 17c connected to the CPU 14 with its memory 14c and the clinical software of the device provide electromagnetic noise filters, and gain adjustable by the examining physician.

### Plethysmogram and Oximetry (FIG. 20):

FIG. 20 shows that the ECG electrodes have an optical window 19a through which the plethysmograph/oxymeter 19 operates, being able to obtain plethysmogram and electrocardiogram simultaneously. In the same way as with the electrocardiogram, the doctor immediately sees the oxygen saturation value and the plethysmogram generated in his web APP and can modify the sweep speed, gain and filters, as well as require some action from the patient to achieve the best result.

The doctor can also save these data in the patient's medical record if he so wishes.

To determine the plethysmographic curve and oxygen saturation value, the device has a green/red LED pulse emitter 19b and a infrared LED pulse emitter 19c powered by a driver 19d and controlled by the CPU by means of a sequencer 19e of internal pulses that are delivered through the optical window 19a at the fingertip or earlobe of the patient. By means of the measurement of a measurement photosensor 19f and an ambient light photosensor 19g, connected to an analog-digital converter 19h which is in turn connected to the processor 14a, the reflection of the light modulated by the blood vessels is evaluated; they expand and contract as a function of the blood pulse that passes through them. Oxygenated hemoglobin absorbs more infrared light and allows more red light to pass through, while hemoglobin without oxygen absorbs more red light and allows more infrared light to pass through. In this way, by means of the clinical software of the device, the plestysmographic curve and oxygen saturation level SpO2 are obtained. The device also includes an ambient light cancellation system that is measured by the 19g ambient light sensor to improve measurement sensitivity and homogeneity across different skin types. The intensity of the ambient light is injected into another A/D converter 19i to feed the CPU 14a with that information.

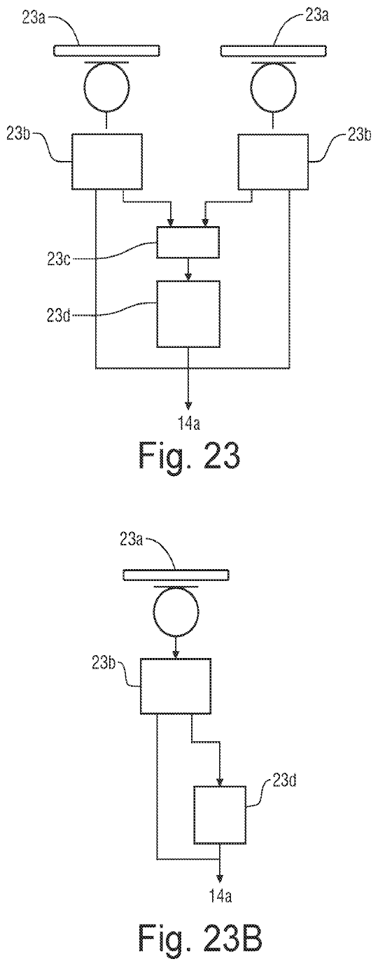

### Digital Electronic Stethoscope for Abdominal, Pulmonary, Cardiological and Peripheral Use (FIG. 10 and FIG. 23):

FIG. 10 shows that the digital electronic stethoscope is located at the opposite end of the pharyngoscope, its bell and membrane protruding in such a way that it can be applied flat and frankly on any part of the body. The clinical software of the digital electronic otoscope has ambient noise and gain cancellation processing, and adjustable filters for use in pediatric or adult patients, regardless of size, weight or ethnicity. The doctor can adjust these processes through his web application according to the progress of the examination and his criteria.

As in any face-to-face consultation, but with more beneficial elements, the doctor can not only modify the auscultation points as necessary in view of numerous cardiac pathologies or anatomies distorted by thoracic malformations or previous surgeries, but can also modify the intensity of the perceived sound as well as apply special filters to better perceive murmurs, heart sounds, vascular murmurs, abdominal auscultation or auscultation of different respiratory phenomena. Different filters are required to optimally auscultate a first heart sound, an aortic murmur, or a murmur of mitral stenosis or a ventricular septal defect.

The present invention foresees that the doctor who uses the digital electronic stethoscope may find himself in a different environment than the patient, so he might not hear environmental noises as he would in face-to-face auscultation and with the mechanisms of the auditory system that allow choosing what a signal to hear, as they are diminished. For this reason, the clinical software of the digital electronic stethoscope of the present invention includes a noise canceling sensor and noise canceling DSP processor.

FIG. 23 shows that both digital acoustic transducers 23a with a large dynamic range of 24Bits, one for measurement and the other for environmental intake, each inject a respective programmable gain amplifier 23b. The outputs of each programmable gain amplifier are connected to a multiplexer 23c which in turn sends the selected signal to an A/D converter 23d. These signals are processed by DSP software with noise cancellation, selective filters for different organs, and adjustable gain to fine-tune and optimize listening. All these parameters are controlled and adjusted by the doctor from their application in order to obtain an optimal result.

An alternative stethoscope configuration with a single acoustic transducer is seen in FIG. 23A.

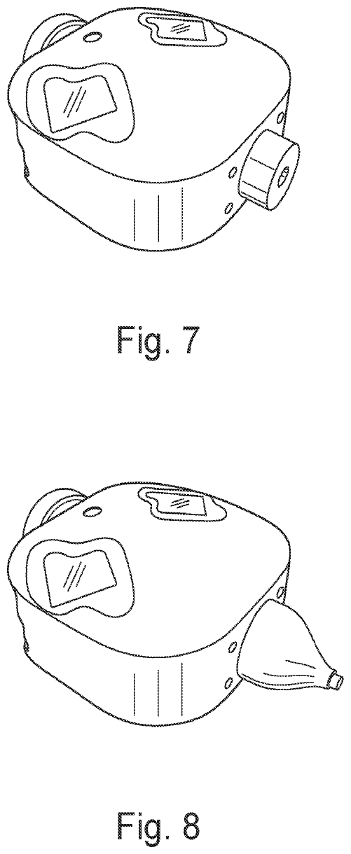

### Pharyngoscope (FIGS. 7 and 22).

FIG. 7 shows the pharyngoscope used for throat examination. It has a camera with illumination of light intensity with controlled spectrum. The clinical device takes high-speed images during image visualization and centering, and is sent at full resolution once the physician observes the target lesion or pathology.

In this case, the patient himself or a family member, in the case of children, aim the pharyngoscope of the device supporting the lower tongue provided for this purpose in order to make a good visualization of the required area as indicated by the doctor. Simultaneously, the doctor sees the patient through the camera and the cell phone APP provides visual and verbal indications through the cell phone APP and views the image obtained at the same time on the screen of their APP. The doctor also controls the lighting level from his web APP. The device also has a removable, sanitizable tongue depressor.

FIG. 22 shows that the hardware of the device has a camera 20d with an optical lens 20c and an LED 20a to provide illumination powered by an LED driver 20b. Both, the camera 20d and the LED driver 20b are connected to the processor 14a and are controlled by the physician by means of clinical software to perform a throat exam with adjustable spectrum compensated illumination.

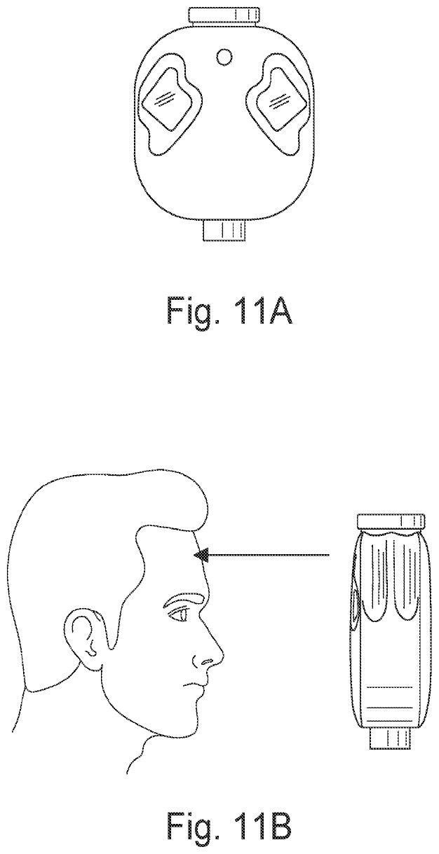

### Infrared Thermometer (FIGS. 11 and 25).

FIGS. 11A and 11B show that, in the upper part, between the ECG electrodes is the infrared sensor for free-of-contact temperature measurement, from a distance between 5 cm and 2 cm. The doctor can indicate different places on the patient to take the temperature. From the doctor's web APP, all the parameters and records made during the interactive remote clinical examination are displayed at the same time they are performed. The doctor is in control of the examination in all cases and may record the information obtained in the patient's medical record if he considers it appropriate.

FIG. 25 shows that the thermometer of the clinical device of the present invention uses a two-area sensor for temperature measurement by infrared emission at a distance of 2 to 5 cm, digitizing the measurement for immediate compensation against ambient temperature, achieving accuracy and repeatability better than 0.2 degrees centigrade. The areas comprise an IR proximity sensor 22a and a room temperature sensor 22b that feed their signals to an A/D converter 22c which sends the data to the CPU 14a.

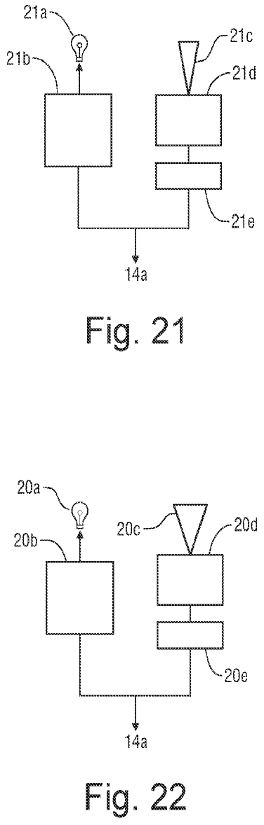

### Otoscope: (FIGS. 8 and 21)

FIG. 8 shows that the otoscope operates in a similar way to the pharyngoscope with the appropriate size for visualization within the ear canal. It has a lighting that can be adjusted by the doctor for the correct observation of the corresponding areas. It has a support for the placement of disposable conical specula of different sizes, for pediatric or adult use.

FIG. 21 shows that the hardware of the device has a camera 21d with lens 21c and LEDs 21a to provide illumination within the ear canal. Both, the camera 21d through a control interface 21e and the LED driver 21b are connected to the processor 14a and are controlled by the physician through clinical software.

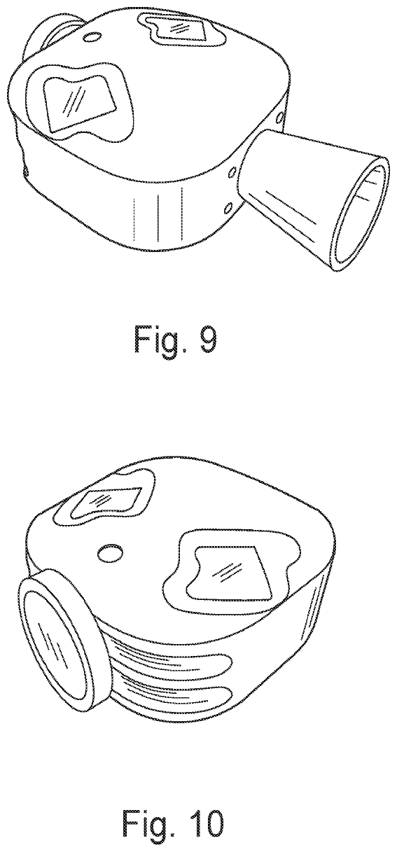

### Dermatoscope: (FIG. 9)

The dermatoscope provides the optics for the adequate visualization of dermatological lesions through the camera with illumination adjustable by the doctor.

The clinical software for the dermatoscope uses the same hardware as the pharyngoscope (FIG. 22). In the dermatoscope, the hardware of the device takes advantage of the camera 20d with an optical lens 20c and the LED 20a to provide illumination powered by an LED driver 20b. Both the camera 20d and the LED driver 20b are connected to the processor 14a and are controlled by the physician through clinical software to perform a skin examination of the patient.

### Real Time Spectrography

In an alternative embodiment, the present invention also comprises an array of emitters and photodetectors of different wavelengths, also including precision temperature sensors and clinical software for analysis by the spectral composition of a test strip placed in the corresponding slot. This mechanism has been designed to perform analysis using test strips, each made to detect and quantify different types of reagents.

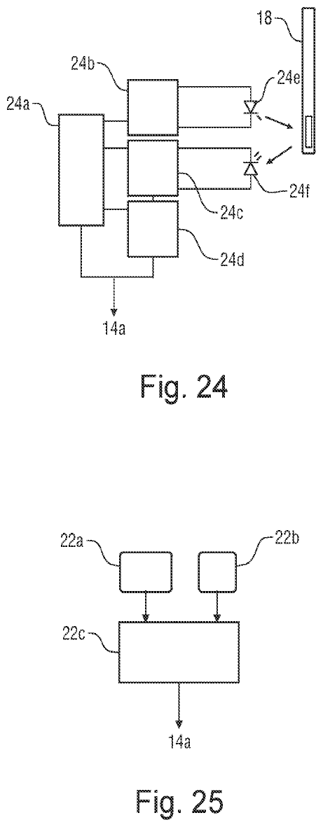

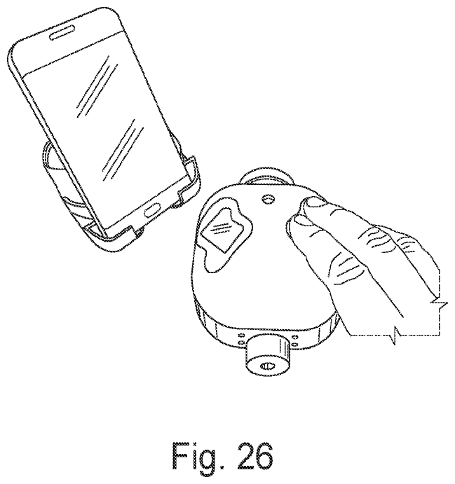

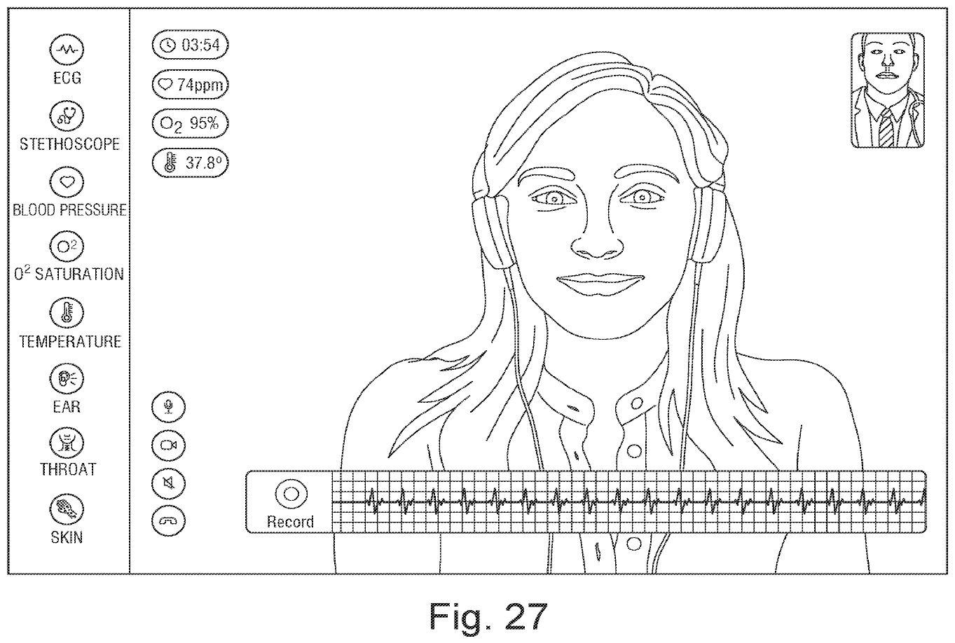

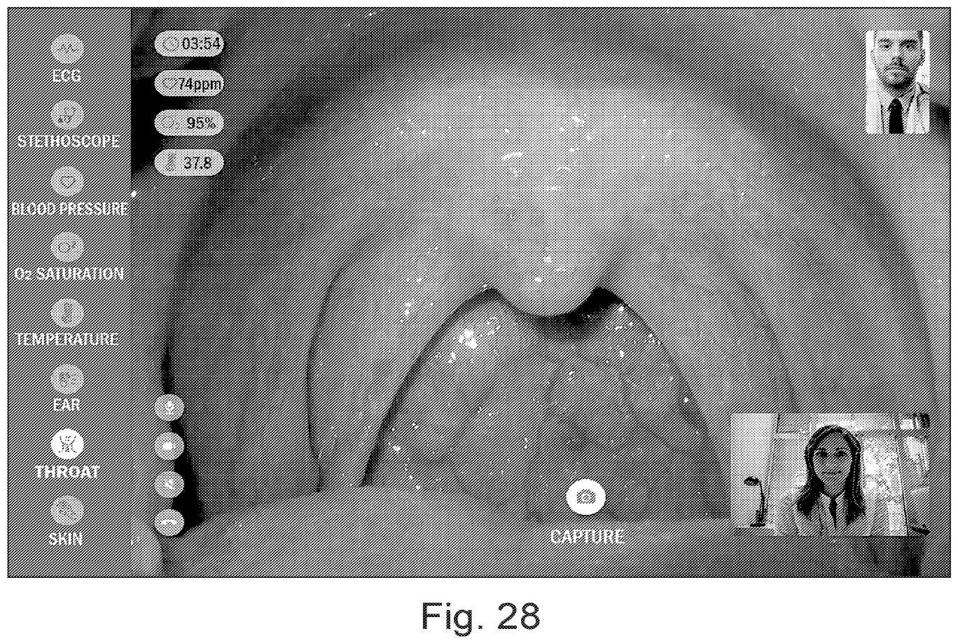

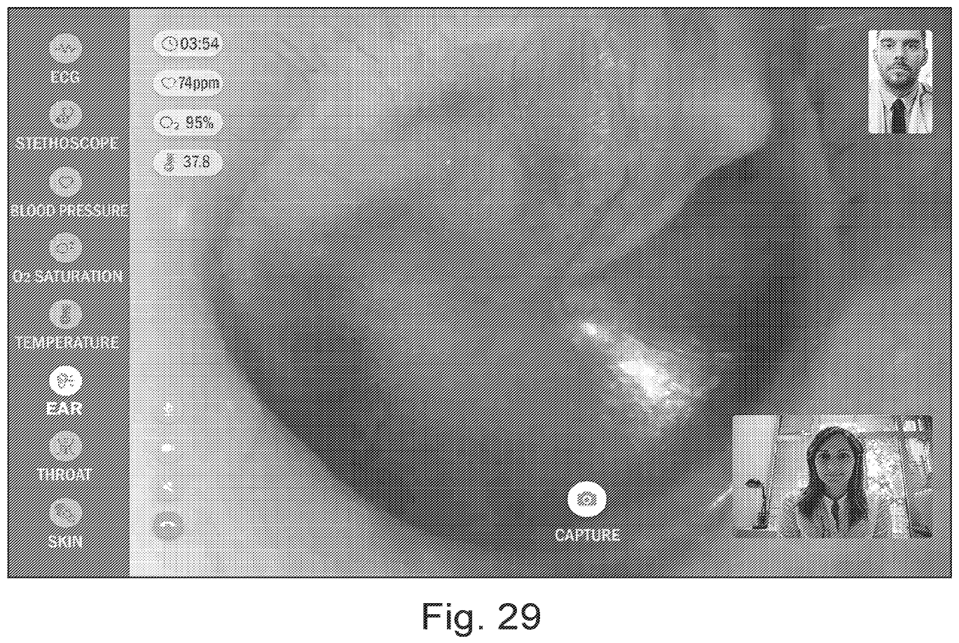

FIG. 24 shows that the hardware of the IRCEPD 6 has a sequencer 24a that controls the driver 24b of a broad spectrum light emitter. This light falls on the test strip sample and the reflected light is taken by an array of multispectral light receivers 24f connected to a programmable signal amplifier 24c which in turn performs the corresponding temperature correction. The signal is connected to the analog to digital converter 24d from where the data is sent to the processor 14a of the unit to be evaluated by the clinical software of the device.

### Other Optional Clinical Instruments

In another alternative embodiment, the IRCEPD device comprises a blood pressure meter derived from the oximeter that is incorporated therein.

In a further alternative embodiment, the IRCEPD device comprises a blood pressure meter connected to it by bluetooth.

In a further alternative embodiment, the IRCEPD device comprises a spirometer incorporated therein.

In a further alternative embodiment, the IRCEPD device comprises a spirometer connected to it by bluetooth.

In a further alternative embodiment, the IRCEPD device comprises a bilirubinometer incorporated therein.

In a further alternative embodiment, the IRCEPD device comprises an abdominal or cardiac ultrasound transducer incorporated therein.

### Internal Rechargeable Battery:

FIGS. 12A and 12B show that the electronics of the remote clinical platform device (DEPECRI) 6 is completely powered by an energy management circuit 13a that provides through a voltage regulator 13b the different internal voltages required by the circuits from a 13d battery. The battery 13d is charged via a wireless coupler 13c at the base of the clinical device so that it is in charging mode while not in use and is thus ready for immediate use. Also, in this way the clinical device is not connected at any time to the electrical network, thus guaranteeing electrical safety.

### ANNEX OF REFERENCES

1. Doctors and health professionals conducting consultation.
2. Front end on the doctor's computer, notebook, tablet or cell phone
3. Internet cloud.
4. Backend for the management of the interactive office.
5. Management of shifts, users etc.
6. Interactive remote clinical examination platform device (IRCEPD).
7. Clinical instruments.
8. Patient.
9. Peer to Peer Communications.
10. Cell phone of the patient with APP for interaction with instruments and doctor.
11. Additional interconsultation professionals
12. Platform device accessories
13a. Power manager
13b. Voltage regulator
13c. Wireless battery charger
13d. LiOn battery
14a. CPU
14b. Operating system
14c. Software
14d. Memory
14e. Data bus
15a. WiFi-Bluetooth® circuit
15b. WiFi-BlueTooth® antenna
16. Expansion bus internal instruments
17. Electrocardiograph
17a. Electrocardiograph (ECG) electrodes
17b. High impedance preamp and programmable gain
17c. A/D converter
18. Test strip
19. Plethysmograph and oximeter
19a. Plethysmograph optical window
19b. Red/green emitter led
19c. Infrared emitter LED
19d. LED driver
19e. Pulse sequencer
19f. Photosensor
19g. Ambient light photo sensor
19h. Measurement photosensor A/D converter
19i. Environmental Photo Sensor A/D Converter
20. Pharyngoscope and dermatoscope
20 a. Pharyngoscope LED Light
20b. Pharyngoscope lighting control driver
20c. Pharyngoscope optics
20d. Pharyngoscope camera
20e. Pharyngoscope control interface
21. Otoscope
21a. Otoscope LED Light
21b. Otoscope lighting control driver
21c. Otoscope optics
21d. Otoscope camera
21e. Otoscope control interface
22. Clinical thermometer (non-contact)
22a. Thermometer IR proximity sensor
22b. Ambient temperature sensor
22c. A/D converter
23. Environmental noise canceling digital electronic stethoscope.
23a. Membrane of the stethoscope
23b. Programmable gain amplifier
23c. Multiplexor
23d. A/D converter
24. Reflection Spectrometer-Spectroscopy Test Strip Analyzer.
24a. Sequencer
24b. LED driver
24c. Amplifier, corrector and spectrum selector
24d. A/D converter
24e. Wide spectrum light emitting LED
24f. Multi-spectrum light receiver
25. Authentication request call and call cut-off.
26. Hospital communication endpoint (OMBU API Endpoint).
27. Private access session communication.
28. Endpoint of communication at patient site.

Embodiments of components of the systems described herein might be coupled directly or indirectly to memory elements through a system bus such as a data, address, and/or control bus. The memory elements can include local memory employed during actual execution of the program code, bulk storage, and cache memories which provide temporary storage of at least some program code in order to reduce the number of times code must be retrieved from bulk storage during execution.

It should also be noted that at least some of the operations for the methods may be implemented using software instructions stored on a computer useable storage medium for execution by a computer. As an example, an embodiment of a computer program product includes a computer useable storage medium to store a computer readable program that, when executed on a computer, causes the computer to perform operations, including an operation to monitor a pointer movement in a web page. The web page displays one or more content feeds. In one embodiment, operations to report the pointer movement in response to the pointer movement comprising an interaction gesture are included in the computer program product. In a further embodiment, operations are included in the computer program product for tabulating a quantity of one or more types of interaction with one or more content feeds displayed by the web page.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

Embodiments of the invention can take the form of an entirely hardware embodiment, an entirely software embodiment, or an embodiment containing both hardware and software elements. In one embodiment, the invention is implemented in software, which includes but is not limited to firmware, resident software, microcode, etc.

Furthermore, embodiments of the invention can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

Input/output or I/O devices (including but not limited to keyboards, displays, pointing devices, etc.) can be coupled to the system either directly or through intervening I/O controllers. Additionally, network adapters also may be coupled to the system to enable the data processing system to become coupled to other data processing systems or remote printers or storage devices through intervening private or public networks. Modems, cable modems, and Ethernet cards are just a few of the currently available types of network adapters.

Additionally, some or all of the functionality described herein might be implemented via one or more controllers, processors, or other computing devices. For example, a controller might be implemented to control the mooring lines, the tether(s) or tendon(s), or modes of the system.

In the above description, specific details of various embodiments are provided. However, some embodiments may be practiced with less than all of these specific details. In other instances, certain methods, procedures, components, structures, and/or functions are described in no more detail than to enable the various embodiments of the invention, for the sake of brevity and clarity.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

Although specific embodiments of the invention have been described and illustrated, the invention is not to be limited to the specific forms or arrangements of parts so described and illustrated. The scope of the invention is to be defined by the claims appended hereto and their equivalents.

1. An interactive remote clinical examination platform device (IRCEPD) comprising a power management unit, a rechargeable battery, a CPU, a memory, an external communication WiFi circuit and a WiFi and BlueTooth® antenna, and an internal instrument expansion circuit, comprising a group of instruments formed by, but not limited to:
an electrocardiograph ECG;
a plethysmograph and pulse oximeter;
a pharyngoscope and dermatoscope;
an otoscope;
a contact-free clinical thermometer; and
a digital electronic stethoscope.

2. The interactive remote clinical examination platform device (IRCEPD) according to claim 1, further comprising a test strip analyzer spectrometer for rapid analysis by real-time spectroscopy.

3. The interactive remote clinical examination platform device (IRCEPD) according to claim 1, wherein said power management unit comprises a wireless battery charger and a voltage regulator.

4. The interactive remote clinical examination platform device (IRCEPD) according to claim 1, wherein said EGC electrocardiograph comprises a pair of electrodes connected to a programmable gain high impedance preamplifier, a plurality of filters and an A/D converter.

5. The interactive remote clinical examination platform device (IRCEPD) according to claim 1, wherein said plethysmograph and pulse oximeter comprises an emitter for generating green/red LED pulsed light and an emitter for generating LED infrared pulsed light, both fed by a driver and controlled by the CPU by means of an internal pulse sequencer, and an optical window through which said pulsed light and said pulsed infrared light are applied to a patient; and wherein the device further comprises a measurement photosensor and a first A/D converter for the photosensor.

6. The interactive remote clinical examination platform device (IRCEPD) according to claim 5, further comprising an ambient light photosensor that provides an ambient light cancellation system to increase sensitivity and homogeneity measurement through different types of skin, and wherein the intensity of the ambient light level signal is injected to the CPU via a second A/D converter.

7. The interactive remote clinical examination platform device IRCEPD according to claim 5, wherein, for the determination of a plethysmographic curve and oxygen saturation value of a patient, the device is capable of measuring the reflection of light modulated by the patient's blood vessels that expand and contract depending on the blood pulse that passes through them and, based on the fact that oxygenated hemoglobin absorbs more infrared light and allows more red light to pass while hemoglobin without oxygen absorbs more red light and allows more infrared to pass through, the device is capable of obtaining the plethysmographic curve and SpO2 oxygen saturation level.

8. The interactive remote clinical examination platform device (IRCEPD) according to claim 1, wherein said pharyngoscope and dermatoscope comprises a camera and an optics and a LED (20a) to provide lighting powered by an LED driver, and wherein the camera and the LED driver are connected to the processor to perform a throat examination with adjustable illumination of compensated spectrum.

9. The interactive remote clinical examination platform device (IRCEPD) according to claim 1, wherein said an otoscope comprises a camera, a lens for ear examination with a disposable and sanitizable removable speculum, and a LED to provide adjustable illumination of compensated spectrum inside the ear canal, and wherein the camera through a control interface and the driver of LEDs are connected to the processor.

10. The interactive remote clinical examination platform device (IRCEPD), according to claim 1, wherein said clinical thermometer comprises an infrared proximity body temperature sensor and a room temperature sensor, both connected to a third A/D converter.

11. The interactive remote clinical examination platform device (IRCEPD) according to claim 1, wherein said digital electronic stethoscope comprises environmental noise cancellation processors, with gain and adjustable filters for auscultation with specialized filters.

12. The interactive remote clinical examination platform device (IRCEPD), according to claim 11, wherein said specialized filters of said electronic stethoscope are applied to auscultate selected organs from a group consisting of: heart, abdomen, lungs, vascular tracts.

13. The interactive remote clinical examination platform device (IRCEPD) according to claim 2, wherein said test strip analyzer spectrometer comprises a sequencer, a wide spectrum LED light emitter fed by a driver, a plurality of photosensors of different wavelengths calibrated to conform a spectral analysis and that feed their signal to a correction amplifier and with spectrum selection which is connected to an A/D converter that sends the data to the CPU.

14. The interactive remote clinical examination platform device IRCEPD, according to claim 1, further comprising a clinical instrument selected from a group consisting of, but not limited to: a blood pressure meter derived from the oximeter and contained inside the IRCEPD; a blood pressure meter connected to the IRCEPD via Bluetooth®; a spirometer contained inside the IRCEPD; a spirometer connected to the IRCEPD vía Bluetooth®; a bilirubinometer contained inside the IRCEPD; and an abdominal or cardiac ultrasound transducer contained inside the IRCEPD.

15. An interactive remote clinical examination platform assembly, comprising:
a) an interactive remote clinical examination platform device IRCEPD;
b) a wireless charging base;
c) a cell phone in a support base so that the patient can interact with the doctor with image and sound with the doctor without occupying his hands;
d) a set of accessories for the instruments of the IRCEPD; and
e) a dermatoscope adapter and an otoscope adapter.

16. The interactive remote clinical examination platform assembly according to claim 15, further comprising: f) reagent strips for rapid analysis in the real-time spectroscope.

17. The interactive remote clinical examination platform assembly, according to claim 15, further comprising a clinical instrument selected from a group consisting of, but not limited to: a blood pressure meter derived from the oximeter and contained inside the IRCEPD; a blood pressure meter connected to the IRCEPD via Bluetooth®; a spirometer contained inside the IRCEPD; a spirometer connected to the IRCEPD via Bluetooth®; a bilirubinometer contained inside the IRCEPD; an abdominal or cardiac ultrasound transducer contained inside the IRCEPD.

18. A remote clinic arrangement for conducting interactive medical clinical examinations comprising an interactive remote clinical examination device (IRCEPD), comprising:
(a) a patient-site platform assembly comprising said interactive remote clinical examination device (IRCEPD), and instrument accessories;
b) a first front end computing device for a doctor to enter a remote clinical office session in real time, to use the device, to consult and record the patient's medical history;
c) a cell phone equipped with a management and communication application APP for a patient; and
d) a back end computing device for data processing and storage of the digital medical record.

19. The arrangement according to claim 18, wherein said first frontend computing device is capable of communicating over the Internet with cloud servers (3) to request P2P (peer to peer) signaling, verify labor shifts, obtain authentication and have access to a patient's medical history through an Ipad, a Tablet, a laptop or a smartphone, or through the application APP to communicate with at least one IRCEPD.

20. The arrangement according to claim 18, further comprising a second front end device at a site distant from said doctor for interconsultation with other professionals sharing the information of the patient interactively, allowing the discussion and analysis of the case simultaneously or carrying out a medical conference

21. (canceled)

22. (canceled)

23. (canceled)
